(19) United States
(12) Patent Application Publication
(10) Pub. No.: US 2007/0208309 A1
(43) Pub. Date: Sep. 6, 2007

(54) Endcap for a Vacuum Lancing Fixture

(76) Inventors: Bruce A. Flora, Goshen, IN (US); Donald R. Hesser, Decatur, MI (US); George V. Siddons, Elkhart, IN (US)

Correspondence Address: BAYER HEALTHCARE, LLC, 555 WHITE PLAINS ROAD, TARRYTOWN, NY, 10591, US

(21) Appl. No.: 10/592,342

(22) PCT Filed: Apr. 1, 2005
(86) PCT No.: PCT/US05/11340

§ 371 Date: Sep. 11, 2006

Related U.S. Application Data
(60) Provisional application No. 60/558,243, filed on Apr. 1, 2004.

(51) Int. Cl. A61M 37/00

(52) U.S. Cl. 604/140

(57) ABSTRACT

An endcap for a vacuum-assisted lancing device has a contoured skin-contact surface for placement against a skin surface. The skin-contact surface has a contour that gradually progresses from a skin-contact edge toward an inner wall of the endcap, with the diameter of the skin-contact surface at the skin-contact edge being greater than the diameter of the skin-contact surface at the inner wall of the endcap.

### FIELD OF THE INVENTION

The present invention is generally directed to skin-lancing devices and is more specifically directed to a skin lancing device having an improved endcap for more effective lancing and more efficient vacuum formation.

### BACKGROUND OF THE INVENTION

Lancing devices are used for obtaining capillary blood from body sites. A typical user of a lancing device is a person in a program of self-blood-glucose-monitoring for treatment of diabetes. Such a user presses an endcap of the lancing device at a selected puncture site, activates the lancing device to puncture the skin at the site, and draws capillary blood for testing. To minimize any discomfort caused by the puncture, the lancing device typically controls the depth of the puncture, quickly withdraws the lancet from the skin once a puncture has been made, and prevents the lancet from rebounding and reentering the puncture or causing a second puncture.

Once a puncture has been made, the lancing device remains on the site and a vacuum is created to draw skin partially into an end cap of the device. As this occurs, a small amount of blood forms on the skin at the puncture site. The vacuum is then released and the lancing device is removed from the skin. The drop of blood on the surface of the skin at the puncture site is then applied to a test sensor.

Some lancing devices include a gasket connected to the lancet that reciprocates in the lancing device as the lancet moves through a lancing stroke. The gasket is in airtight contact with the inside of the lancing device such that as the gasket slides within the lancing device, air is displaced and a vacuum is created.

It is desirable that lancing devices be as comfortable as possible to use. Further, it is desirable for a lancing device to have accurate placement and puncture depth for the lance, so that an appropriate amount of blood collects with only one lancing operation, decreasing or eliminating the possibility that a second lancing operation will be necessary. Additionally, it is desirable for a lancing device to create and maintain sufficient vacuum for a drop of blood of a predictable volume to collect. The present invention is directed to an improved lancing device endcap.

### SUMMARY OF THE INVENTION

An endcap according to one embodiment of the present invention is provided with a contoured skin-contact surface to facilitate the drawing of skin into the endcap

The contour of an endcap according to the present invention preferably includes a narrowing skin-receiving area that causes skin to bulge within the skin-receiving area, preparing the skin for puncturing by a lance to a desired puncture depth.

Increased contact between skin and a lancing device endcap according to some embodiments of the present invention increases the effectiveness and duration of vacuum formation within a vacuum cavity.

Additional features of the present invention will be more fully understood upon reference to the figures.

### BRIEF DESCRIPTION OF THE DRAWINGS

FIG. 1 is a side view of a lancing device according to one embodiment of the present invention.

FIG. 2 is a side cutaway view of an endcap for a lancing device according to one embodiment of the present invention.

FIGS. 3a-3d are time elapse images of use of a lancing device having an endcap according to one embodiment of the present invention.

While the invention is susceptible to various modifications and alternative forms, specific embodiments are shown by way of example in the drawings and will be described in detail herein. However, it should be understood that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE ILLUSTRATED EMBODIMENTS

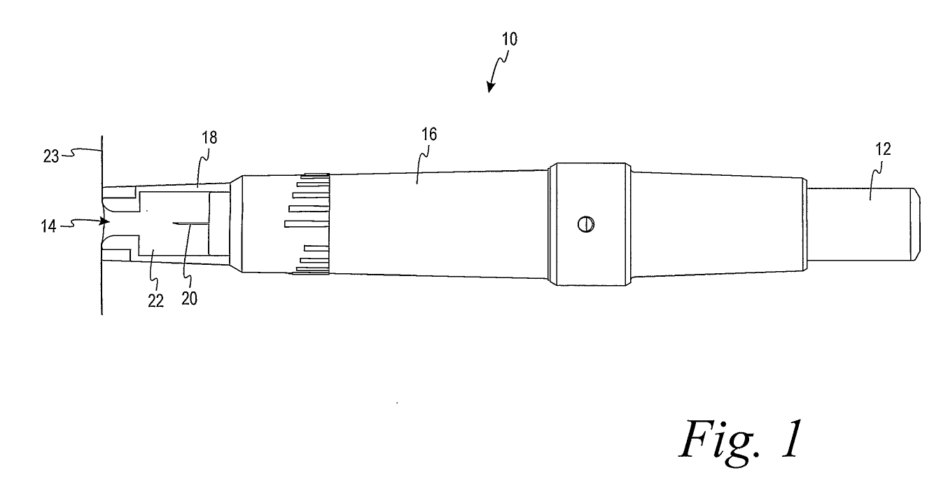

Referring now to FIG. 1, a vacuum-assisted lancing device 10 is shown according to one embodiment of the present invention. A vacuum member, such as a diaphragm (not shown), within the lancing device 10 is activated when the plunger 12 is depressed by the user and travels toward the open end 14 of the lancing device 10. As the plunger 12 is depressed, a rebound spring (not shown) within the housing 16 of the lancing device 10 is expanded and extended and the diaphragm (not shown) is displaced toward the endcap 18.

As the plunger 12 is depressed farther, a lance 20, such as a sterile needle lance, quickly extends out of the open end 14 of the lancing device 10 and is then quickly retracted within the endcap 18. Following the lancing operation, a vacuum is formed within a vacuum cavity 22 by the movement of the diaphragm within the lancing device 10. The vacuum cavity 22 is bounded by the inner surfaces of the endcap 18 and by the surface 23 of skin against which the endcap 18 is placed. Further details of operation of lancing devices according to some embodiments of the present invention will be understood with reference to U.S. Pat. No. 6,152,942 which is incorporated by reference herein in its entirety. According to an alternative embodiment, a puncturing process starts when sufficient pressure has been exerted between the endcap and the skin surface 23 to initiate the puncture.

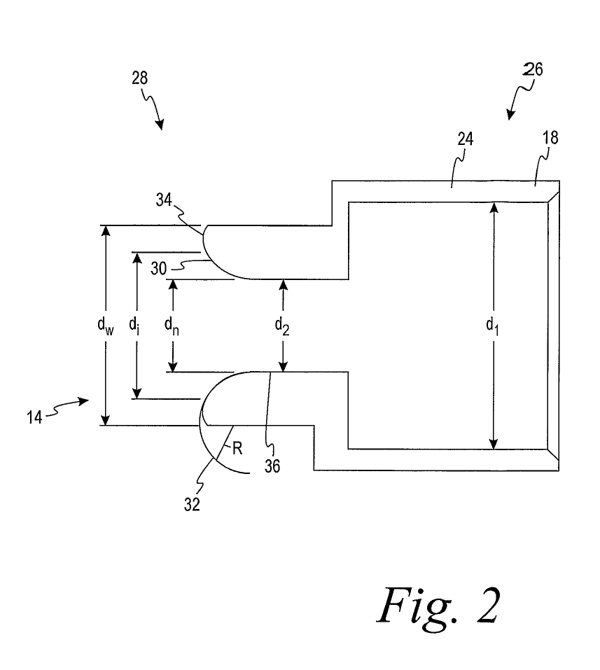

The endcap 18 of the present invention is provided with features that improve the performance of the lancing device 10. FIG. 2 is a cutaway side view of an endcap 18 according to one embodiment of the present invention. The endcap 18 is provided with a cylindrical cavity wall 24 at a first portion 26 of the endcap 18. The first portion 26 of the endcap attaches to the housing 16 of the lancing device (shown in FIG. 1). The first portion 26 of the endcap 18 may attach to the housing 16 of the lancing device in a variety of ways (for example, via friction-fit, snap-on, or screw-on connections). The endcap 18 is attached to the housing so that a vacuum may be maintained within the internal volume of the endcap. An O-ring, for example, may be placed between the endcap and an endcap-mating surface of the housing 16 of the lancing device to result in creation and maintenance of a substantially airtight connection allowing formation of a vacuum within the internal volume of the endcap. It is preferable for a mating surface between the endcap 18 and the housing 16 to allow for adjustment of puncture depth.

The second portion 28 of the endcap 18 is narrower than the first portion 26 of the endcap 18. A skin-contact surface 30 slopes inwardly from an outer wall 32 along a blunt skin-contact edge 34. The skin-contact surface 30 is widest at the skin-contact edge 34 and gradually narrows from the open end 14 of the lancing device to an inner wall 36. According to one embodiment of the present invention, the skin-contact surface 30 follows a parabolic contour from the skin-contact edge 34 to a linear inner wall 36. Other contours, such as circular contours, may be used to form the skin-contact surface 30. In the embodiment shown in FIG. 2, the skin-contact surface has a cross-section that approximates an arc of a circle having a radius R of approximately 3.89 mm. According to other embodiments, the radius R may range from approximately 1.5 mm to approximately 6 mm.

As shown in FIG. 2, the inner volume of the first portion 26 of the endcap 18 has a diameter d₁ and the inner volume of the second portion 28 of the endcap has a diameter d₂. According to one embodiment of the present invention, the diameter d₁ of the inner volume of the first portion 26 of the endcap is approximately 19 mm and the diameter d₂ of the inner volume of the second portion 28 of the endcap is approximately 7 mm. The skin-contact surface 30 narrows from a widest diameter d_{w} at the skin-contact edge 34, through an intermediate diameter dᵢ to a narrowest diameter dₙ at the inner wall 36 of the second portion 28 of the endcap. According to one embodiment of the present invention, the widest diameter d_{w} is approximately 13 mm, the intermediate diameter dᵢ is approximately 10 mm, and the narrowest diameter dₙ is approximately 7 mm. It is to be understood that larger or smaller diameters may be employed in other embodiments of the present invention. Smaller diameters, such as the diameter shown in FIG. 2, are preferred in some embodiments because they allow for lancing to be done as fingertips as well as other sites, such as the forearms. Wider minimum diameters, such as approximately 10 mm or 13 mm, for the narrowest diameter dₙ may be used, as may smaller diameters such as approximately 5 mm. According to some embodiments, diameters dₙ of from approximately 7 mm to approximately 10 mm are used.

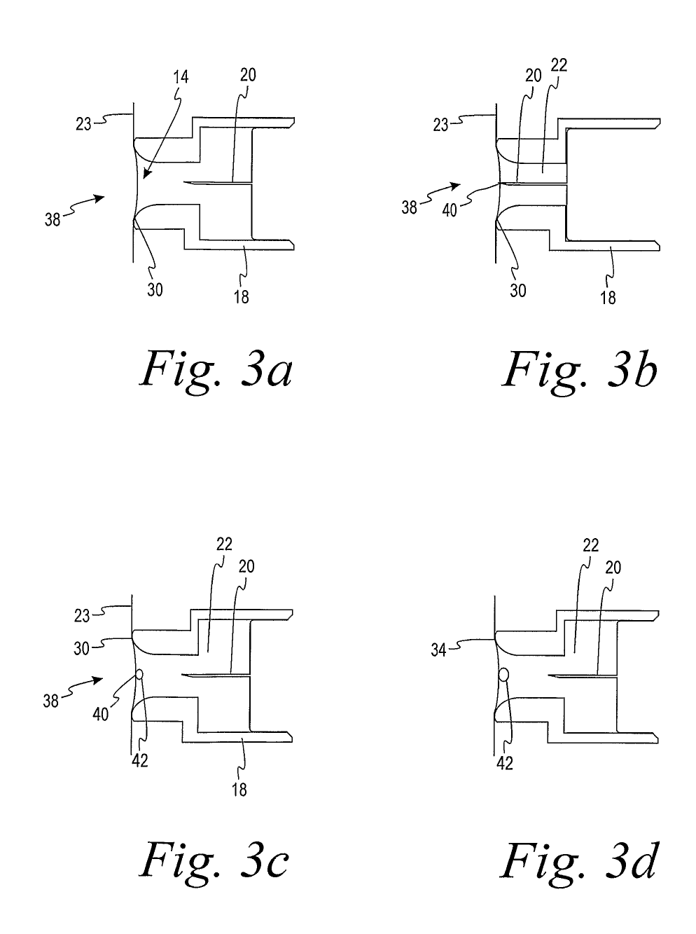

The operation of an endcap 18 according to the present invention will be more completely understood with reference to FIGS. 3a to 3d, which show a time-elapse progression of a puncturing operation. In FIG. 3a, the endcap 18 has been placed against a skin surface 23, resulting in a bulge 38 of the skin surface 23 due to pressure against the skin surface 23. The contour of the skin-contact surface 30 causes bunching up of the skin surface 23 within the open end 14 of the endcap 18, positioning the skin surface 23 for lancing by the lance 20. The lance 20 has not yet been driven toward the skin surface 23 in FIG. 3a.

Turning to FIG. 3b, the lance 20 has been driven beneath the skin surface 23 at a puncture site 40 where the greatest depth of skin has entered the vacuum cavity 22 of the endcap 18. The contour of the skin-contact surface 30 provides a predictable and repeatable bunching of skin within the vacuum cavity 22. As a result, the puncture depth to which the lance 20 is driven beneath the skin surface 23 is easily controlled and kept at approximately the same depth with repeated lancing operations. Puncture depth is controlled to reduce pain and injury at the site. According to some embodiments, puncture depths between approximately 1 mm and 2 mm are preferred.

In FIG. 3c, the lance 20 has been withdrawn from the skin surface 23 and a vacuum is formed within the vacuum cavity 22. As a result, the bulge 38 of the skin surface 23 has increased and a blood sample 42 has begun to collect at the puncture site 40. The vacuum facilitates blood flow at the puncture site 40. Because the contour of the skin-contact surface 30 approximately matches the contour of the skin surface 23 as it forms the bulge 38, the vacuum formed within the vacuum cavity 22 will have lower pressure, more reliability, and longer duration than a vacuum formed with an endcap that makes less contact with the skin surface 23. Finally, as shown in FIG. 3d, the vacuum is maintained within the vacuum cavity 22 and the blood sample 42 continues to grew in size following retraction of the lance 20. Throughout the process, the rounded skin-contact edge 34 increases comfort for the user as compared to endcaps having sharper angles. This is important in embodiments in which pressure is placed on the skin to create and maintain a vacuum within the endcap.

Endcaps according to the present invention are capable of maintaining a vacuum pressure of −10 mm Hg for six seconds or longer. Further, the control of puncture depth limits the amount of pain and injury associated with the puncture while providing an adequate blood sample. Reduced pain and injury promotes increased testing and the improved feedback results in better control of blood sugar.

Endcaps according to the present invention may be made of a variety of materials. It is preferable for endcaps according to the present invention to be transparent, enabling a user to view the collection of a blood sample 42 and to withdraw the endcap from the skin surface when a sufficient volume of blood has been collected. It is preferred for endcaps according to the present invention to be disposable and easily attached to and removed from a lancing device.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the claimed invention, which is set forth in the following claims.

1. An endcap for a vacuum-assisted lancing device comprising:
a contoured skin-contact surface for placement against a skin surface, said skin-contact surface having a contour that gradually progresses from a skin-contact edge toward an inner wall of the endcap, a diameter of said skin-contact surface at said skin-contact edge being greater than a diameter of said skin-contact surface at said inner wall of said endcap.

2. The endcap of claim 1 wherein said endcap comprises first and second portions, said first portion of said endcap having a greater diameter than said second portion of said endcap and being adapted for connection to a housing of said vacuum-assisted lancing device, said skin-contact surface being provided on said second portion of said endcap.

3. The endcap of claim 1 in which said contour of said skin-contact surface is a circular arc contour.

4. The endcap of claim 3 wherein a radius of said circular arc is between approximately 1.5 mm and approximately 6 mm.

5. The endcap of claim 1 wherein said contour of said skin-contact surface is a parabolic contour.

6. The endcap of claim 1 wherein said contour of said skin-contact surface causes controlled bulging of skin when said skin-contact surface is depressed against a skin surface.

7. The endcap of claim 1 wherein said endcap is transparent.

8. The endcap of claim 2 wherein an inner radius of said second portion is between approximately 5 mm and approximately 13 mm.

9. A method of collecting a blood sample for testing, the method comprising the acts of:
providing a vacuum-assisted lancing device having a lance and an endcap, said endcap having a contoured skin-contact surface progressing from a first diameter at a skin-contact edge to a second diameter at an inner wall of said endcap, said first diameter being greater than said second diameter, said skin-contact surface surrounding an open end of said endcap, said endcap defining at least a portion of said vacuum cavity;
pressing said open end of said endcap against a skin surface;
forming a bulge on said skin surface via the interaction of said skin-contact surface and said skin surface;
puncturing said skin surface at a puncture site of said bulge with said lance; and
forming a vacuum within said vacuum cavity such that a blood sample collects at said puncture site.

10. The method of claim 9 wherein the acts are performed in the order listed.

11. The method of claim 9 wherein said skin-contact surface of said endcap has a circular arc contour progressing from said first diameter to said second diameter.

12. The method of claim 11 wherein said circular arc has a radius of from approximately 1.5 to approximately 6 mm.

13. The method of claim 9 wherein said skin-contact surface of said endcap has a parabolic contour progressing from said first diameter to said second diameter.

14. The method of claim 9 wherein said endcap comprises a first portion for attachment to said vacuum-assisted lancing device and a second portion for contacting skin.

15. The method of claim 9 further comprising maintaining said vacuum at a pressure of approximately −10 mm Hg for at least approximately six seconds.

16. Amended) A vacuum-assisted lancing device comprising:
a housing;
a lance;
an endcap connected to said housing at a substantially airtight interface, said endcap comprising a first portion for connection to said housing and a second portion for skin contact, said second portion of said endcap having an inner wall and a skin-contact surface, said skin-contact surface surrounding an open end of said endcap, said skin-contact surface having a contour and progressing from a first diameter at said open end of said endcap to a second diameter where said skin-contact surface meets said inner wall.

17. The vacuum-assisted lancing device of, claim 16 wherein said contour of said skin-contact surface is an arc of a circle.

18. The vacuum-assisted lancing device of claim 17 wherein a radius of said arc is from approximately 1.5 mm to approximately 6 mm.

19. The vacuum-assisted lancing device of claim 16 wherein said contour of said skin-contact surface is parabolic.

20. The vacuum-assisted lancing device of claim 16 wherein said endcap is adapted to maintain a vacuum of approximately −10 mm Hg for at least approximately 6 seconds during use of said vacuum-assisted lancing device.
